(19) United States
(12) Patent Application Publication
(10) Pub. No.: US 2013/0116737 A1
(43) Pub. Date: May 9, 2013

(54) Methods for Assessing Renal Neuromodulation Treatment and Associated Systems and Methods

(71) Applicant: Midtronic Ardian Luxembourg S.a.r.l., Luxembourg (LU)

(72) Inventors: Susan Edwards, Santa Rosa, CA (US); Howad R. Levin, Teaneck, NJ (US); Julie Trudel, Santa Rosa, CA (US)

(73) Assignee: MEDTRONIC ARDIAN LUXEMBOURG S.A.R.L., Luxembourg (LU)

(21) Appl. No.: 13/670,374

(22) Filed: Nov. 6, 2012

Related U.S. Application Data
(60) Provisional application No. 61/556,711, filed on Nov. 7, 2011.

(51) Int. Cl. A61N 1/36

(52) U.S. Cl. 607/2

(57) ABSTRACT

The present technology relates to methods for assessing renal neuromodulation treatment and associated systems and methods. In particular, various embodiments of the present technology relate to assessing the efficacy of an ongoing or completed renal neuromodulation procedure and providing feedback (e.g., visual and/or audible indications) to a clinician with the results of all or part of such procedures.

### TECHNICAL FIELD

The present technology relates generally to methods for assessing renal neuromodulation treatment and associated systems and methods. In particular, several embodiments are directed to approaches for evaluating the efficacy of intravascular renal neuromodulation and for conveying such information as procedural feedback and/or diagnostic information.

### BACKGROUND

The sympathetic nervous system (SNS) is a primarily involuntary bodily control system typically associated with stress responses. Fibers of the SNS innervate tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (such as heart failure), and progressive renal disease. For example, radiotracer dilution has demonstrated increased renal norepinephrine (NE) spillover rates in patients with essential hypertension.

Cardio-renal sympathetic nerve hyperactivity can be particularly pronounced in patients with heart failure. For example, an exaggerated NE overflow from the heart and kidneys to plasma is often found in these patients. Heightened SNS activation commonly characterizes both chronic and end stage renal disease. In patients with end stage renal disease, NE plasma levels above the median have been demonstrated to be predictive for cardiovascular diseases and several causes of death. This is also true for patients suffering from diabetic or contrast nephropathy. Evidence suggests that sensory afferent signals originating from diseased kidneys are major contributors to initiating and sustaining elevated central sympathetic outflow.

Sympathetic nerves to the kidneys terminate in the blood vessels, the juxtaglomerular apparatus, and the renal tubules. Stimulation of the renal sympathetic nerves can cause increased renin release, increased sodium (Na⁺) reabsorption, and a reduction of renal blood flow. These neural regulation components of renal function are considerably stimulated in disease states characterized by heightened sympathetic tone and likely contribute to increased blood pressure in hypertensive patients. The reduction of renal blood flow and glomerular filtration rate as a result of renal sympathetic efferent stimulation is likely a cornerstone of the loss of renal function in cardio-renal syndrome (i.e., renal dysfunction as a progressive complication of chronic heart failure). Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). These pharmacologic strategies, however, have significant limitations including limited efficacy, compliance issues, side effects, and others. Accordingly, there is a strong public-health need for alternative treatment strategies.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates a renal neuromodulation system configured in accordance with an embodiment of the present technology.

FIG. 2 illustrates modulating renal nerves with a catheter apparatus in accordance with an embodiment of the technology.

FIG. 3 is a block diagram illustrating a method for providing feedback to a clinician in accordance with an embodiment of the present technology.

FIGS. 4-6 illustrate representative generator display screens configured in accordance with aspects of the present technology.

FIG. 7 is a conceptual illustration of the sympathetic nervous system (SNS) and how the brain communicates with the body via the SNS.

FIG. 8 is an enlarged anatomic view of nerves innervating a left kidney to form the renal plexus surrounding the left renal artery.

FIGS. 9A and 9B provide anatomic and conceptual views of a human body, respectively, depicting neural efferent and afferent communication between the brain and kidneys.

FIGS. 10A and 10B are, respectively, anatomic views of the arterial and venous vasculatures of a human.

### DETAILED DESCRIPTION

The present technology is directed to methods, systems, and apparatuses for providing feedback related to renal neuromodulation procedures (i.e., rendering neural fibers that innervate the kidney inert or inactive or otherwise completely or partially reduced in function) by percutaneous transluminal intravascular access. In particular, embodiments of the present technology relate to methods, systems, and apparatuses that assess the efficacy of an ongoing or completed renal neuromodulation procedure and provide feedback conveying the success of all or part of such procedures. In certain embodiments, one or more visual cues may be displayed to provide the feedback. Such visual cues can include, but are not limited to, text messages or descriptors, numeric calculated values or measures, or other forms of alphanumeric fields that represent the feedback information. In other embodiments, other types of visual feedback may be provided instead of (or in addition to) alphanumeric feedback. For example, in such other embodiments, one or more color or graphical indications may be displayed that convey the desired feedback information without using text or numeric characters. In yet further embodiments, an audible indication (e.g., an audible beep, chime, or tone) may be provided in addition to or in lieu of a visual indication to convey the desired feedback information.

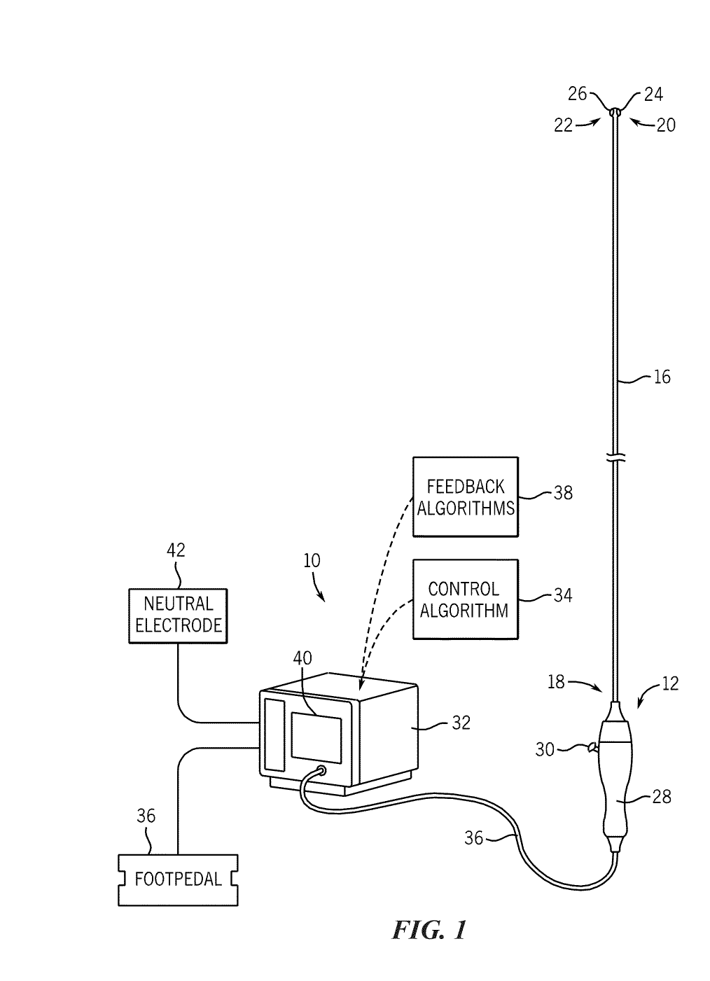

Specific details of several embodiments of the technology are described below with reference to FIGS. 1-10B. Although many of the embodiments are described below with respect to methods, systems, and devices for evaluating neuromodulation treatment, other applications and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-10B.

The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

### I. Renal Neuromodulation

Renal neuromodulation is the partial or complete incapacitation or other effective disruption of nerves innervating the kidneys. In particular, renal neuromodulation comprises inhibiting, reducing, and/or blocking neural communication along neural fibers (i.e., efferent and/or afferent nerve fibers) innervating the kidneys. Such incapacitation can be long-term (e.g., permanent or for periods of months, years, or decades) or short-term (e.g., for periods of minutes, hours, days, or weeks). Renal neuromodulation is expected to efficaciously treat several clinical conditions characterized by increased overall sympathetic activity, and in particular conditions associated with central sympathetic over stimulation such as hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, and sudden death. The reduction of afferent neural signals contributes to the systemic reduction of sympathetic tone/drive, and renal neuromodulation is expected to be useful in treating several conditions associated with systemic sympathetic over activity or hyperactivity. Renal neuromodulation can potentially benefit a variety of organs and bodily structures innervated by sympathetic nerves. For example, a reduction in central sympathetic drive may reduce insulin resistance that afflicts patients with metabolic syndrome and Type II diabetics. Additionally, osteoporosis can be sympathetically activated and might benefit from the downregulation of sympathetic drive that accompanies renal neuromodulation. A more detailed description of pertinent patient anatomy and physiology is provided in Section II below.

Various techniques can be used to partially or completely incapacitate neural pathways, such as those innervating the kidney. The purposeful application of energy (e.g., electrical energy, thermal energy) to tissue by energy delivery element(s) can induce one or more desired thermal heating effects on localized regions of the renal artery and adjacent regions of the renal plexus RP, which lay intimately within or adjacent to the adventitia of the renal artery. The purposeful application of the thermal heating effects can achieve neuromodulation along all or a portion of the renal plexus RP.

The thermal heating effects can include both thermal ablation and non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature can be above body temperature (e.g., approximately 37° C.) but less than about 45° C. for non-ablative thermal alteration, or the target temperature can be about 45° C. or higher for the ablative thermal alteration.

More specifically, exposure to thermal energy (heat) in excess of a body temperature of about 37° C., but below a temperature of about 45° C., may induce thermal alteration via moderate heating of the target neural fibers or of vascular structures that perfuse the target fibers. In cases where vascular structures are affected, the target neural fibers are denied perfusion resulting in necrosis of the neural tissue. For example, this may induce non-ablative thermal alteration in the fibers or structures. Exposure to heat above a temperature of about 45° C., or above about 60° C., may induce thermal alteration via substantial heating of the fibers or structures. For example, such higher temperatures may thermally ablate the target neural fibers or the vascular structures. In some patients, it may be desirable to achieve temperatures that thermally ablate the target neural fibers or the vascular structures, but that are less than about 90° C., or less than about 85° C., or less than about 80° C., and/or less than about 75° C. Regardless of the type of heat exposure utilized to induce the thermal neuromodulation, a reduction in renal sympathetic nerve activity (“RSNA”) is expected. A more detailed description of pertinent patient anatomy and physiology is provided in Section IV below.

### II. Systems and Methods for Renal Neuromodulation

FIG. 1 illustrates a renal neuromodulation system 10 (“system 10”) configured in accordance with an embodiment of the present technology. The system 10 includes an intravascular treatment device 12 operably coupled to an energy source or energy generator 32. In the embodiment shown in FIG. 1, the treatment device 12 (e.g., a catheter) includes an elongated shaft 16 having a proximal portion 18, a handle assembly 28 at a proximal region of the proximal portion 18, and a distal portion 20 extending distally relative to the proximal portion 18. The treatment device 12 further includes a therapeutic assembly or treatment section 22 including one or more energy delivery elements 24 (e.g., electrode(s)) at or near the distal portion 20 of the elongated shaft 16. In the illustrated embodiment, one or more additional electrode(s) 26 may be provided at or near the treatment section 22 to provide measurements or data that may be used in evaluating various aspects of the neuromodulation treatment as whole or of the formation of individual, discrete lesions formed as part of the treatment. In other embodiments, the energy delivery element 24 may be an electrode configured to both deliver energy as part of the neuromodulation treatment and to provide measurements or data used to evaluate the success of lesion formation and/or of the overall neuromodulation treatment. In still further embodiments, the therapeutic assembly or treatment section 22 may comprise a multi-electrode array including one or more additional energy delivery elements 24.

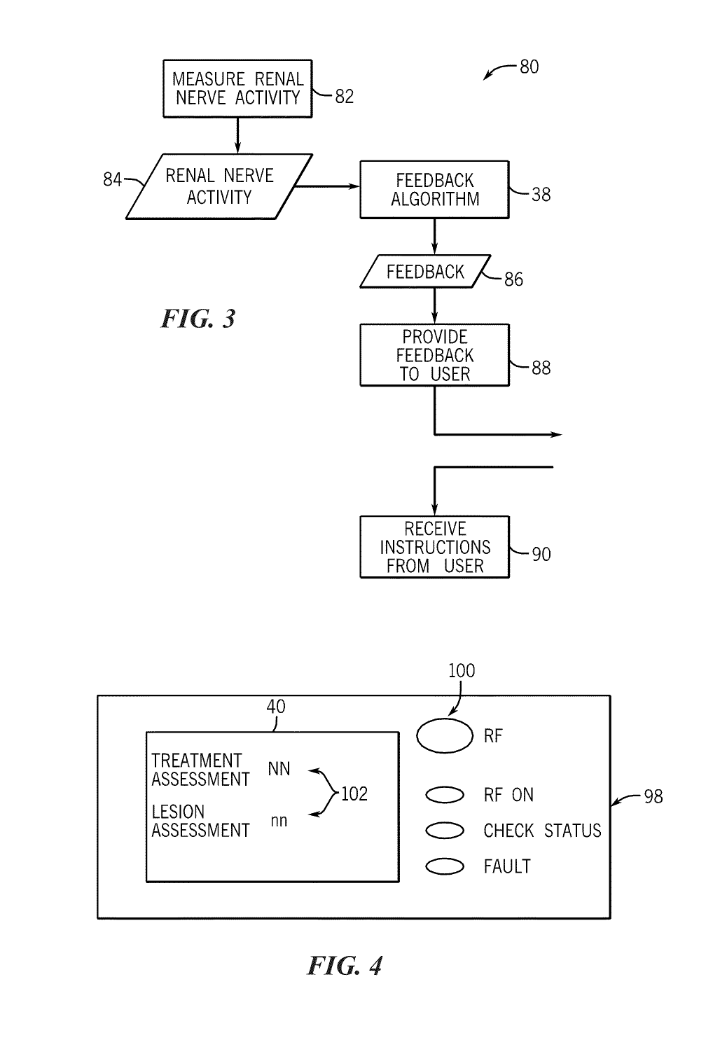

The energy generator 32 (e.g., a RF energy generator) is configured to generate a selected form and magnitude of energy for delivery to the target treatment site via the energy delivery element(s) 24. The energy generator 32 can be electrically coupled to the treatment device 12 via a cable 36. At least one supply wire (not shown) passes along the elongated shaft 16 or through a lumen in the elongated shaft 16 to the energy delivery element 24 and transmits the treatment energy to the energy delivery element 24. A control mechanism, such as foot pedal 36, may be connected (e.g., pneumatically connected or electrically connected) to the energy generator 32 to allow the operator to initiate, terminate and, optionally, adjust various operational characteristics of the energy generator, including, but not limited to, power delivery. The energy generator 32 can be configured to deliver the treatment energy via an automated control algorithm 34 and/or under the control of a clinician. In addition, one or more feedback algorithms 38 may be executed on a processor of the system 10. Such feedback algorithms 38, when executed in conjunction with a treatment operation, may provide feedback to a clinician of the system 10, such as via a display 40 associated with the system 10. The feedback or evaluation may allow an operator of the system 10 to determine the success of a given treatment and/or to evaluate possible failure conditions. This feedback, therefore, may be useful in helping the operator learn how to increase the likelihood of success when performing a treatment. Further details regarding suitable control algorithms 34 and feedback algorithms 38 are described below with reference to FIGS. 3-6.

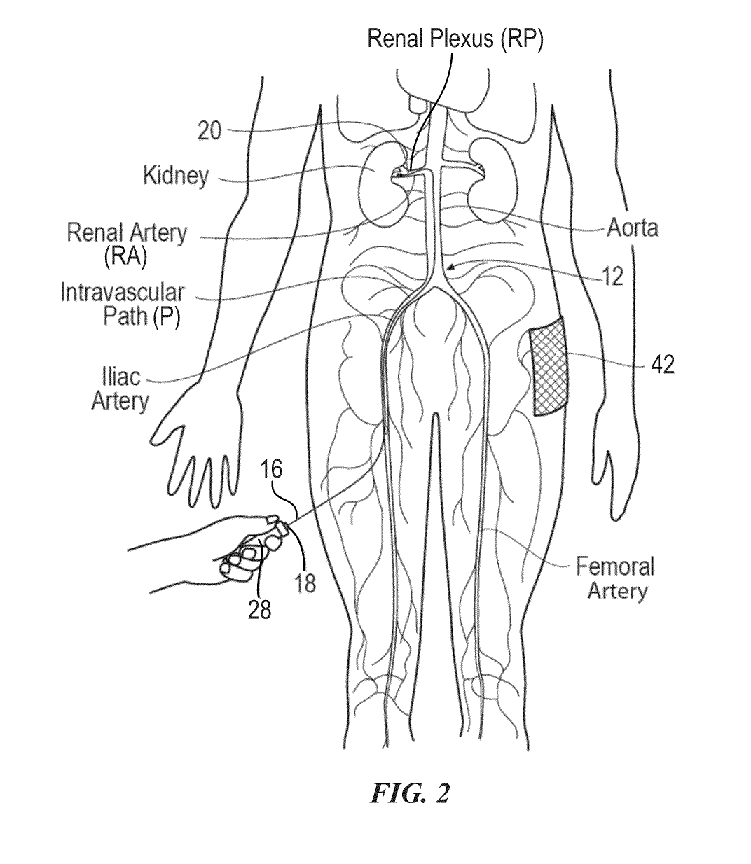

In some embodiments, the system 10 may be configured to provide delivery of a monopolar electric field via the energy delivery element 24. In such embodiments, a neutral or dispersive electrode 42 may be electrically connected to the energy generator 32 and attached to the exterior of the patient (as shown in FIG. 2). Additionally, one or more sensors (not shown), such as one or more temperature (e.g., thermocouple, thermistor, etc.), impedance, pressure, optical, flow, chemical or other sensors, may be located proximate to or within the energy delivery element 24 and connected to one or more of the supply wires (not shown). For example, a total of two supply wires may be included, in which both wires could transmit the signal from the sensor and one wire could serve dual purpose and also convey the energy to the energy delivery element 24. Alternatively, both wires could transmit energy to the energy delivery element 24.

In embodiments including multiple energy delivery elements 24, the energy delivery elements 24 may deliver power independently (i.e., may be used in a monopolar fashion), either simultaneously, selectively, or sequentially, and/or may deliver power between any desired combination of the elements (i.e., may be used in a bipolar fashion). Furthermore, the clinician optionally may be permitted to choose which energy delivery element(s) 24 are used for power delivery in order to form highly customized lesion(s) within the renal artery, as desired.

The computing devices on which the system 10 is implemented may include a central processing unit, memory, input devices (e.g., keyboard and pointing devices), output devices (e.g., display devices), and storage devices (e.g., disk drives). The output devices may be configured to communicate with the treatment device 12 (e.g., via the cable 36) to control power to the energy delivery element 24 and/or to obtain signals from the energy delivery element 24 or any associated sensors. Display device(s) (e.g., the display 40) may be configured to provide indications of power levels or sensor data, such as audio, visual or other indications, or may be configured to communicate the information to another device.

The memory and storage devices are computer-readable media that may be encoded with computer-executable instructions that implement the object permission enforcement system, which means a computer-readable medium that contains the instructions. In addition, the instructions, data structures, and message structures may be stored or transmitted via a data transmission medium, such as a signal on a communications link and may be encrypted. Various communications links may be used, such as the Internet, a local area network, a wide area network, a point-to-point dial-up connection, a cell phone network, and so on.

Embodiments of the system 10 may be implemented in and used with various operating environments that include personal computers, server computers, handheld or laptop devices, multiprocessor systems, microprocessor-based systems, programmable consumer electronics, digital cameras, network PCs, minicomputers, mainframe computers, computing environments that include any of the above systems or devices, and so on.

The system 10 may be described in the general context of computer-executable instructions, such as program modules, executed by one or more computers or other devices. Generally, program modules include routines, programs, objects, components, data structures, and so on that perform particular tasks or implement particular abstract data types. Typically, the functionality of the program modules may be combined or distributed as desired in various embodiments.

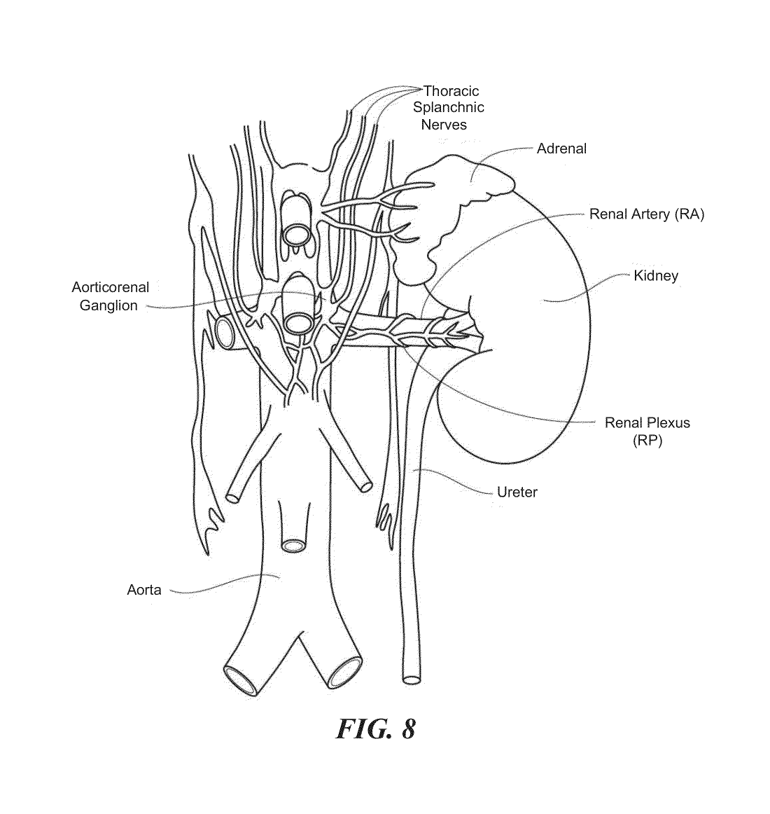

FIG. 2 (and with reference to FIG. 8) illustrates modulating renal nerves with an embodiment of the system 10 shown in FIG. 1. The treatment device 12 provides access to the renal plexus RP through an intravascular path, such as from a percutaneous access site in the femoral (illustrated), brachial, radial, or axillary artery to a targeted treatment site within a respective renal artery RA. As illustrated, a section of the proximal portion 18 of the shaft 16 is exposed externally of the patient. By manipulating the proximal portion 18 of the shaft 16 from outside the intravascular path (e.g., via the handle assembly 28), the clinician may advance the shaft 16 through the sometimes tortuous intravascular path and remotely manipulate or actuate the distal portion 20 of the shaft 16. Image guidance, e.g., computed tomography (CT), fluoroscopy, intravascular ultrasound (IVUS), optical coherence tomography (OCT), or another suitable guidance modality, or combinations thereof, may be used to aid the clinician's manipulation. Further, in some embodiments, image guidance components (e.g., IVUS, OCT) may be incorporated into the treatment device 12 itself. Once proximity between, alignment with, and contact between the energy delivery element 24 (FIG. 1) and tissue are established within the respective renal artery, the purposeful application of energy from the energy generator 32 (FIG. 1) to tissue by the energy delivery element 24 induces one or more desired neuromodulating effects on localized regions of the renal artery and adjacent regions of the renal plexus RP, which lay intimately within, adjacent to, or in close proximity to the adventitia of the renal artery. The purposeful application of the energy may achieve neuromodulation along all or a portion of the renal plexus RP.

The neuromodulating effects are generally a function of, at least in part, power, time, contact between the energy delivery element(s) 24 and the vessel wall, and blood flow through the vessel. The neuromodulating effects may include denervation, thermal ablation, and non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature may be above body temperature (e.g., approximately 37° C.) but less than about 45° C. for non-ablative thermal alteration, or the target temperature may be about 45° C. or higher for the ablative thermal alteration. Desired non-thermal neuromodulation effects may include altering the electrical signals transmitted in a nerve.

### III. Evaluation of Renal Neuromodulation Treatment

A. Overview

In one implementation, a treatment administered using the system 10 constitutes delivering energy through one or more energy delivery elements (e.g., electrodes) to the inner wall of a renal artery for a predetermined amount of time (e.g., 120 sec). Multiple treatments (e.g., 4-6) may be administered in both the left and right renal arteries to achieve the desired coverage. A technical objective of a treatment may be, for example, to heat tissue to a desired depth (e.g., at least about 3 mm) to a temperature that would lesion a nerve (e.g., about 65° C.). A clinical objective of the procedure typically is to neuromodulate (e.g., lesion) a sufficient number of renal nerves (either efferent or afferent nerves of the sympathetic renal plexus) to cause a reduction in sympathetic tone. If the technical objective of a treatment is met (e.g., tissue is heated to about 65° C. to a depth of about 3 mm) the probability of forming a lesion of renal nerve tissue is high. The greater the number of technically successful treatments, the greater the probability of modulating a sufficient proportion of renal nerves, and thus the greater the probability of clinical success.

In certain embodiments, the efficacy of a neuromodulation procedure may be assessed by one or more approaches, such as using injectable substances, mechanical stimuli, electrical stimulation, or other stimuli suitable for generating different types and/or quanta of response when a renal nerve is intact compared to when the renal nerve has been completely or partially inactivated. In certain implementations, it is expected that the desired differential response may be elicited in a clinically useful period of time, such as in less than one or two minutes or, in other implementations, in less than five or ten minutes. Likewise, in such implementations, the differential response may be available at the location where the procedure is performed. In particular, where the differential response is observable at the location where the procedure is performed and/or within a suitable timeframe, the operator may be able to deliver additional therapy (e.g., additional lesion operations) or determine the sufficiency of the delivered therapy based on the observed response.

In certain embodiments, the differential response observed between intact renal nerves and partially or completely incapacitated renal nerves may be provided as a qualitative and/or quantitative input to one or more feedback algorithms 38, as discussed above. The one or more feedback algorithms 38 may be executed on a processor-based component of the system 10, such as a monitor provided with the generator 32. In such implementations, the one or more feedback algorithms 38 may be able to provide a clinician with useful feedback in a simplified form that can be used in evaluating a given neuromodulation treatment or step in such a treatment.

FIG. 3, for example, is a block diagram illustrating a method 80 for providing feedback to a clinician in accordance with an embodiment of the present technology. In this embodiment, some measure 84 of renal nerve activity is generated (block 82) and provided as an input to a feedback algorithm 38. The feedback algorithm 38 in turn generates some indication of feedback 86 to be provided (block 88) to a clinician. For example, the feedback 88 may be an indication (such as a quantitative score or a qualitative assessment) related to the efficacy of a renal neuromodulation treatment or related to the efficacy of one operation or component of such a treatment. In response to the provided feedback 86, a clinician may provide additional instructions (block 90), such as an instruction to continue or extend the renal neuromodulation treatment. In circumstances where the treatment is extended or continued, renal nerve activity may be measured (block 82) again at a subsequent point and additional feedback 86 provided to the clinician.

B. Methods of Assessment

In certain embodiments, the assessment of the procedural or technical success may be based on direct or indirect activation of the efferent and/or afferent renal nerves and direct or indirect measurement of the outcome of this stimulation in the generation of a physiological and/or clinical event/reflex. That is, the assessment of renal neuromodulation success may have two components: an input stimulation of the renal nerves of interest and an output measurement describing activity of the renal nerves of interest. One example of this may include the use of a standard neurostimulator to provide electrical stimulation of the renal artery along with a standard clinical or physiological output measurement.

As will be appreciated, in different embodiments the stimulation and/or measurement may be performed after completion of a treatment protocol or may be performed at different points within the protocol, such as after creation of each lesion or a series of lesions. For example, in embodiments where renal nerve activity is to be assessed after each lesion is created, the diagnostic method comprises stimulating nerve activity in the area of the lesion just created and measurements made on the appropriate side of the lesion for the stimulus used. For example, if a stimulus was made to activate the efferent renal nerves (proximal to the lesion), then a measurement should be made on the opposite (distal) side of the lesion in an area where the specific nerve fibers from proximal to the lesion would be on the distal side of the lesion.

With respect to stimulation of the renal nerves, a variety of approaches may be used to directly stimulate one or both of the efferent and afferent renal nerves in vivo. Examples of such direct stimulation approaches may include, but are not limited to: unilateral or bilateral ureteral occlusion (afferent); electrical stimulation (afferent or efferent) of one or more of the sympathetic ganglia, the vessel wall proximal to a lesion, the vessel wall distal to a lesion, and/or the renal capsule; mechanoreceptor activation (afferent) of one or more of the renal artery, the renal vein, and/or the renal pelvis; or chemoreceptor activation (afferent) such as in the form of a renal artery occlusion sufficient to result in ischemia or a renal pelvis saline infusion. As will be appreciated, the preceding discussion generally relates to non-pharmacological approaches for directly stimulating the renal nerves. In other embodiments, pharmacological approaches may be used in addition to or in place of such non-pharmacological approaches.

Alternatively, a variety of approaches may be used to indirectly stimulate one or both of the efferent and afferent renal nerves in vivo. These stimuli cause activation of the efferent renal nerves as they come from systemic changes. For example, most of the interventions described result in a decrease in cardiac output leading to decreases in blood pressure that are sensed by the aortic and/or carotid baroreceptors. These receptors send feedback to the brain to increase central sympathetic nervous system outflow to the kidney. Chemoreceptor and somatic afferent (e.g., muscle, skin) activation use the same type of afferent mechanism to increase sympathetic nervous system outflow, simply using different receptor types. Examples of such indirect stimulation approaches which increase efferent renal nerve activity may include, but are not limited to: reduction in systemic blood pressure/cardiac output; carotid baroreceptor unloading; nonhypotensive hemorrhage; head-up tilt; cardiac tamponade; positive pressure breathing; systemic chemoreceptor activation (hypoxia and hypercapnia); or somatic afferent activation.

Conversely, interventions that increase blood pressure or vascular volume will decrease sympathetic nervous system outflow. For example, increase in blood volume or inflation of a balloon in the atria to increase atrial wall stress generally results in a reflex decrease in efferent renal nerve activity. Examples of such indirect stimulation approaches that decrease efferent renal nerve activity may include, but are not limited to: increase in systemic blood pressure/cardiac output; atrial distension; head out water immersion; intravascular volume expansion; head-up tilt; or negative pressure breathing. As will be appreciated, the preceding discussion generally relates to non-pharmacological approaches for indirectly stimulating the renal nerves. In other embodiments, however, pharmacological approaches may be used in addition to or in lieu of such non-pharmacological approaches.

In further embodiments, pharmacological (i.e., drug) agents may be used to stimulate, directly or indirectly, one or both of the efferent or afferent renal nerves. Examples of such pharmacological agents may include, but are not limited to: adenosine, nesiritide (BNP), ANP, fenoldopam, bradykinin, capsaicin, angiotensin II, ouabain, phenol, hypo-/hypertonic solutions, hypoxic solutions, phenoxybenzamine/guanethidine, VIP, radiocontrast media, prostaglandin E2, indomethacin, acetylcholine, nitric oxide, norepinephrine, dopamine, amiloride, and cyclosporine. By way of example, activation of renal pelvic chemoreceptors may be accomplished by infusion of adenosine to stimulate the afferent renal nerves. While the preceding list of pharmacological agents represents certain known agents that act to stimulate renal nerve activity, as will be appreciated, any number of infusible agents that stimulate renal nerve activity may conceivably be employed as discussed herein.

The renal nerve activity stimulated by any one of the above approaches (or another suitable approach) may be measured to provide some qualitative and/or quantitative representation of the renal nerve activity that may be processed by a suitable algorithm, such as feedback algorithm 38 as discussed herein. A variety of approaches may be employed to acquire the desired measurements. For example, direct electrical renal nerve stimulation can result in measurable changes in nephron glomerular filtration rate and arteriolar resistance, which may both be measured to provide an indication of renal nerve activity.

By way of further example, for measurement of afferent nerve activity, the entire or a large portion of the kidney may be stimulated to activate enough areas to activate most or all of the afferent nerves. This activity may then be recorded circumferentially via an electrode cuff or recorded at multiple single locations around and/or along the renal artery proximal to the lesion to record afferent nerve activity. Such measurements may be acquired prior to therapy and post-therapy, allowing a ratio of the total activity measured pre- to post-therapy to be determined which may be an accurate measure of afferent denervation.

For measurement of efferent nerve activity, the efferent nerves may be stimulated using one or more of the approaches discussed herein. In one implementation, all of the efferent nerves may be directly stimulated using an electrode cuff to circumferentially stimulate at a location on the renal artery proximal to the lesion where all the efferent nerves have already joined the artery. Measurement of renal nerve activity may then be performed by recording circumferentially via an electrode cuff or at multiple single locations around and/or along the renal artery distal to the lesion.

With the foregoing guidance in mind, the following possible indirect physiological or clinical measures of renal nerve activity are provided by way of example. For example, in one implementation, renal nerve conduction across a lesion may be measured using catheter-based electrical stimulation and recording of nerve activity. In another example, renal tissue norepinephrine may be measured using tissue sampling and assay. In a further example, renin secretion (renal vein or systemic venous) may be measured by withdrawing blood using a catheter placed in the renal or peripheral vein and by assaying the blood. Various biomarkers may be utilized using such techniques including, for example, heat shock proteins (e.g., Hsp70, Hsp70, heme oxygenase 1 (Hmox-1), Hsp90, and many others). In further embodiments, other suitable biomarkers may be used.

In some embodiments, renal blood flow may also measured, such as by using an intrarenal flow “wire,” by noninvasive doppler, and/or by clearance of a contrast agent visible using X-ray imaging. In another embodiment, renal artery pressure (which may increase in response to increased afferent renal nerve activity) may be measured, such as using a micromanometer or fluid filled catheter. Likewise, renal vascular resistance may be measured based on the renal pressure/flow relationship. Similarly, renal artery compliance may be measured based on the renal pressure/volume relationship. In other implementations, diuresis/natriuresis may be measured by collecting urine with a catheter. In addition, glomerular filtration rate may be measured based on insulin clearance or estimated CrCl. Systemic blood pressure may be measured invasively or non-invasively using known approaches. Likewise, heart rate, heart rate variability, and/or QT dispersion may be measured manually or using an electrocardiogram (ECG). In other embodiments, cardiac output may be measured, such as by using thermodilution or other approaches. Likewise, pain, skin blush, and/or temperature may be measured using subjective assays. Lastly, representative hormones may be measured using collected venous blood or other blood from the peripheral vasculature.

C. Providing Feedback to a Clinician

The preceding discussion relates various manners in which electrical, chemical, mechanical, and/or physiological data may be generated and/or measured to assess the success of a lesion generating operation and/or treatment session. As discussed above, this data may be provided as an input to one or more algorithms, such as feedback algorithms 38, and used to generate a visual and/or audible feedback that can be provided to the clinician, thereby allowing the clinician to assess the likely success of a lesion formation operation and/or an overall or partial treatment session.

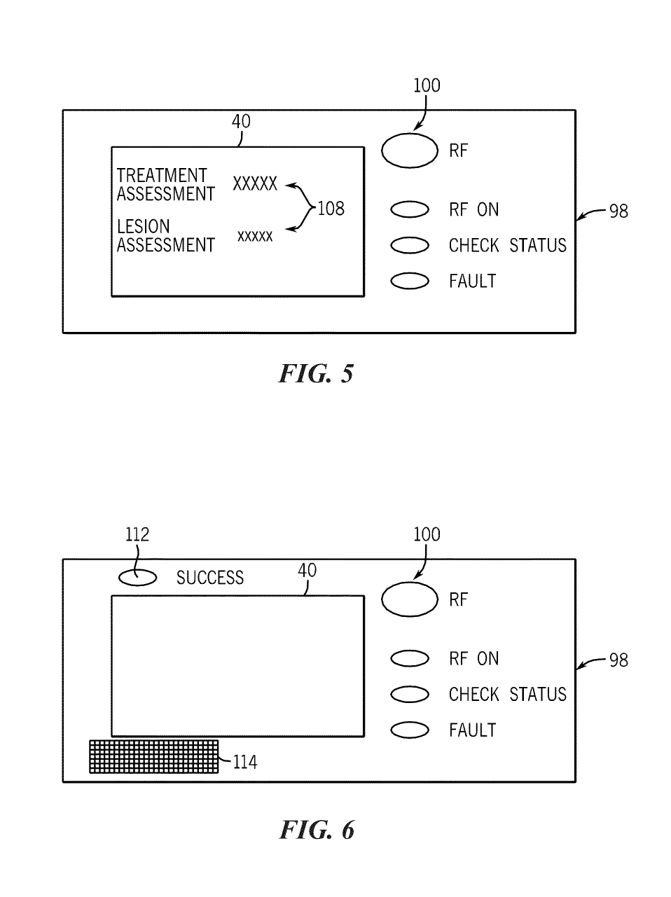

FIG. 4, for example, illustrates a representative display screen or user interface 98 configured in accordance with an embodiment of the present technology. In the depicted example, the user interface 98 may include a variety of indicators that provide information or instructions to the clinician, such as the readiness of the generator 32 (FIG. 1), status warnings or signals, and/or fault indications. In addition, as noted previously, the display 40 can include additional instructions, messages, warnings, and/or information.

In the depicted example, the display 40 may be used to display the output of the feedback algorithm(s) 38 (FIG. 3). For example, in the example illustrated in FIG. 4, one or more numeric indications 102 (such as a percentage or a numeric score) may be provided for an operation used to generate a lesion and/or for an overall or partial renal neuromodulation treatment session. In certain embodiments, the numeric indications 102 may be provided once, such as at the end of an operation or treatment. In other embodiments, the numeric indications 102 may be dynamic or otherwise updatable, such as to provide a running assessment of the activity or functioning of the renal nerves in question.

Referring to FIG. 5, the display screen or user interface 98 may include one or more textual indications 108 informing an operator of the likelihood of success of an operation used to generate a lesion and/or for an overall or partial renal neuromodulation treatment session. As with the numeric indications 102 described above with reference to FIG. 4, the textual indications 108 may be provided once or may be dynamically updated as a treatment session progresses and/or as lesion operations are performed.

Referring next to FIG. 6, the display screen or user interface 98 may include an indicator 112 configured to light up, change color, or otherwise provide an indication that a lesion operation or treatment session is deemed successful or unsuccessful based, at least in part, on feedback generated by the feedback algorithm 38. For example, the indicator 112 may remain unlit to indicate that a treatment did not proceed as planned or lack of success in a treatment or operation or before data is generated one way or another. Upon the feedback algorithm 38 determining that a treatment or operation is successful, however, the indicator 112 may be lit. In other embodiments, the indicator 112 may instead change color to indicate a successful treatment or operation, such as changing from red or yellow to green. As with the preceding examples, an indication provided via the visual indicator 112 may be provided once or may be updated dynamically as a treatment session progresses and/or as lesion operations are performed.

In the embodiment illustrated in FIG. 6, the user interface 98 can also include a speaker 114 configured for playing an audible indication (such as a beep, tone, or audible message). In one or more of the above described embodiments, the described visual indications of treatment success or failure may be accompanied by an audible indication being played on the speaker 114. For example, a respective beep, tone, or message may be played if the feedback algorithm 38 generates an output that a lesion forming operation or a renal neuromodulation treatment was performed unsuccessfully or successfully. In other embodiments, such audio indications may be provided instead of a visual indication. In still further embodiments, various other visual and/or audio indications may be provided to the clinician in addition to, or in lieu of, the various examples described above.

### IV. Pertinent Anatomy and Physiology

The following discussion provides further details regarding pertinent patient anatomy and physiology. This section is intended to supplement and expand upon the previous discussion regarding the relevant anatomy and physiology, and to provide additional context regarding the disclosed technology and the therapeutic benefits associated with renal denervation. For example, as mentioned previously, several properties of the renal vasculature may inform the design of treatment devices and associated methods for achieving renal neuromodulation via intravascular access, and impose specific design requirements for such devices. Specific design requirements may include accessing the renal artery, facilitating stable contact between the energy delivery elements of such devices and a luminal surface or wall of the renal artery, and/or effectively modulating the renal nerves with the neuromodulatory apparatus.

A. The Sympathetic Nervous System

The sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to things as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

1. The Sympathetic Chain

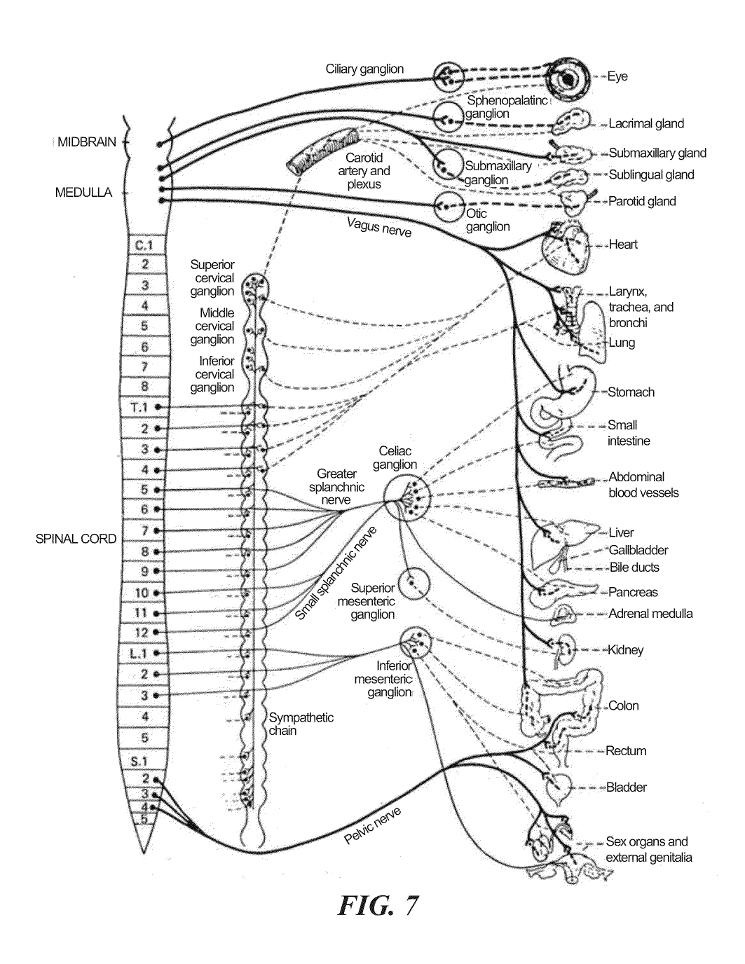

As shown in FIG. 7, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

2. Innervation of the Kidneys

As shown in FIG. 8, the kidney is innervated by the renal plexus RP, which is intimately associated with the renal artery. The renal plexus RP is an autonomic plexus that surrounds the renal artery and is embedded within the adventitia of the renal artery. The renal plexus RP extends along the renal artery until it arrives at the substance of the kidney. Fibers contributing to the renal plexus RP arise from the celiac ganglion, the superior mesenteric ganglion, the aorticorenal ganglion and the aortic plexus. The renal plexus RP, also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, first lumbar splanchnic nerve, second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus RP and are distributed to the renal vasculature.

3. Renal Sympathetic Neural Activity

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); and raise blood pressure. Afferent messages carry signals from various organs and sensory receptors in the body to other organs and, particularly, the brain.

Hypertension, heart failure and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease are characterized by heightened sympathetic nervous activation. In patients with end stage renal disease, plasma levels of norepinephrine above the median have been demonstrated to be predictive for both all-cause death and death from cardiovascular disease. This is also true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well known adverse consequences of chronic sympathetic over activity, such as hypertension, left ventricular hypertrophy, ventricular arrhythmias, sudden cardiac death, insulin resistance, diabetes, and metabolic syndrome.

### (i) Renal Sympathetic Efferent Activity

Sympathetic nerves to the kidneys terminate in the blood vessels, the juxtaglomerular apparatus and the renal tubules. Stimulation of the renal sympathetic nerves causes increased renin release, increased sodium (Na+) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release) and diuretics (intended to counter the renal sympathetic mediated sodium and water retention). However, the current pharmacologic strategies have significant limitations including limited efficacy, compliance issues, side effects and others.

### (ii) Renal Sensory Afferent Nerve Activity

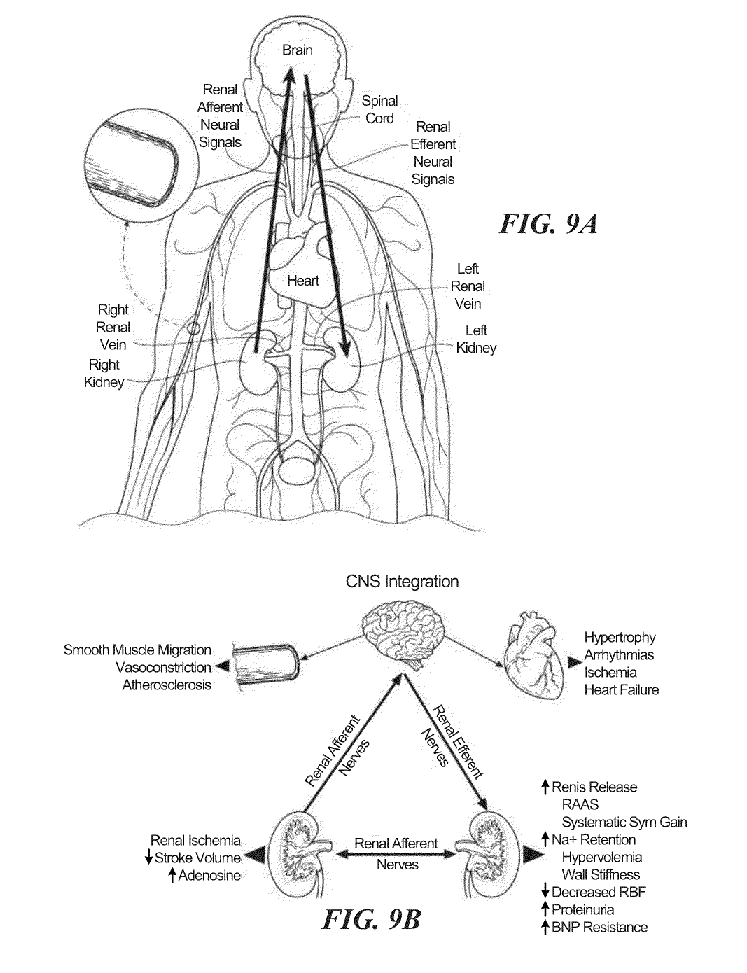

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 9A and 9B, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

B. Additional Clinical Benefits of Renal Denervation

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 7. For example, as previously discussed, a reduction in central sympathetic drive may reduce the insulin resistance that afflicts people with metabolic syndrome and Type II diabetics. Additionally, patients with osteoporosis are also sympathetically activated and might also benefit from the down regulation of sympathetic drive that accompanies renal denervation.

C. Achieving Intravascular Access to the Renal Artery

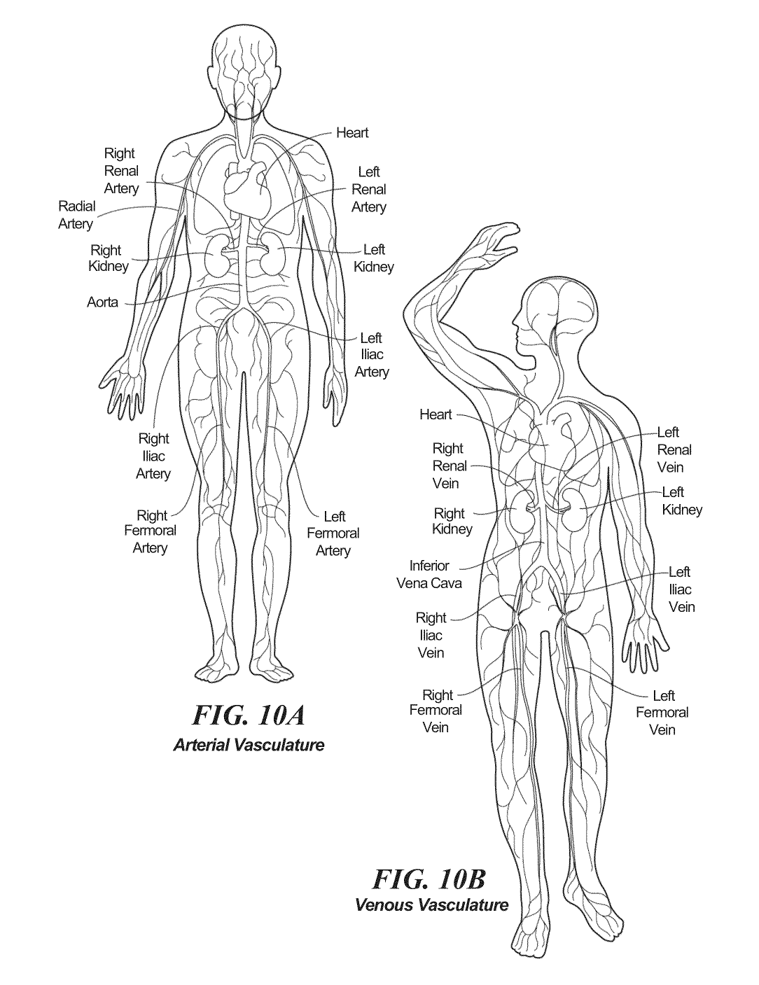

In accordance with the present technology, neuromodulation of a left and/or right renal plexus RP, which is intimately associated with a left and/or right renal artery, may be achieved through intravascular access. As FIG. 10A shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 10B shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique.

D. Properties and Characteristics of the Renal Vasculature

Since neuromodulation of a left and/or right renal plexus RP may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, etc. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. When the neuromodulatory apparatus includes an energy delivery element, such as an electrode, consistent positioning and appropriate contact force applied by the energy delivery element to the vessel wall are important for predictability. However, navigation is impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

Even after accessing a renal artery and facilitating stable contact between neuromodulatory apparatus and a luminal surface of the artery, nerves in and around the adventitia of the artery should be safely modulated via the neuromodulatory apparatus. Effectively applying thermal treatment from within a renal artery is non-trivial given the potential clinical complications associated with such treatment. For example, the intima and media of the renal artery are highly vulnerable to thermal injury. As discussed in greater detail below, the intima-media thickness separating the vessel lumen from its adventitia means that target renal nerves may be multiple millimeters distant from the luminal surface of the artery. Sufficient energy should be delivered to or heat removed from the target renal nerves to modulate the target renal nerves without excessively cooling or heating the vessel wall to the extent that the wall is frozen, desiccated, or otherwise potentially affected to an undesirable extent. A potential clinical complication associated with excessive heating is thrombus formation from coagulating blood flowing through the artery. Given that this thrombus may cause a kidney infarct, thereby causing irreversible damage to the kidney, thermal treatment from within the renal artery should be applied carefully. Accordingly, the complex fluid mechanics and thermodynamic conditions present in the renal artery during treatment, particularly those that may impact heat transfer dynamics at the treatment site, may be important in applying energy (e.g., heating thermal energy) and/or removing heat from the tissue (e.g., cooling thermal conditions) from within the renal artery.

The neuromodulatory apparatus should also be configured to allow for adjustable positioning and repositioning of the energy delivery element within the renal artery since location of treatment may also impact clinical efficacy. For example, it may be tempting to apply a full circumferential treatment from within the renal artery given that the renal nerves may be spaced circumferentially around a renal artery. In some situations, full-circle lesion likely resulting from a continuous circumferential treatment may be potentially related to renal artery stenosis. Therefore, the formation of more complex lesions along a longitudinal dimension of the renal artery may be desirable. The formation of such lesions may be achieved, for example, by repositioning of the neuromodulatory apparatus to multiple treatment locations and/or by using a neuromodulatory apparatus having a mesh structure. Other suitable structures may also be used. It should be noted, however, that a benefit of creating a circumferential ablation may outweigh the potential of renal artery stenosis or the risk may be mitigated with certain embodiments or in certain patients and creating a circumferential ablation could be a goal. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging. Manipulation of a device in a renal artery should also consider mechanical injury imposed by the device on the renal artery. Motion of a device in an artery, for example by inserting, manipulating, negotiating bends and so forth, may contribute to dissection, perforation, denuding intima, or disrupting the interior elastic lamina.

Blood flow through a renal artery may be temporarily occluded for a short time with minimal or no complications. However, occlusion for a significant amount of time should be avoided because to prevent injury to the kidney such as ischemia. It could be beneficial to avoid occlusion all together or, if occlusion is beneficial to the embodiment, to limit the duration of occlusion, for example to 2-5 minutes.

Based on the above described challenges of (1) renal artery intervention, (2) consistent and stable placement of the treatment element against the vessel wall, (3) effective application of treatment across the vessel wall, (4) positioning and potentially repositioning the treatment apparatus to allow for multiple treatment locations, and (5) avoiding or limiting duration of blood flow occlusion, various independent and dependent properties of the renal vasculature that may be of interest include, for example, (a) vessel diameter, vessel length, intima-media thickness, coefficient of friction, and tortuosity; (b) distensibility, stiffness and modulus of elasticity of the vessel wall; (c) peak systolic, end-diastolic blood flow velocity, as well as the mean systolic-diastolic peak blood flow velocity, and mean/max volumetric blood flow rate; (d) specific heat capacity of blood and/or of the vessel wall, thermal conductivity of blood and/or of the vessel wall, and/or thermal convectivity of blood flow past a vessel wall treatment site and/or radiative heat transfer; (e) renal artery motion relative to the aorta induced by respiration, patient movement, and/or blood flow pulsatility: and (f) as well as the take-off angle of a renal artery relative to the aorta. These properties will be discussed in greater detail with respect to the renal arteries. However, dependent on the apparatus, systems and methods utilized to achieve renal neuromodulation, such properties of the renal arteries, also may guide and/or constrain design characteristics.

As noted above, an apparatus positioned within a renal artery should conform to the geometry of the artery. Renal artery vessel diameter, D_{RA}, typically is in a range of about 2-10 mm, with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >5 mm from inner wall of the renal artery) to avoid non-target tissue and anatomical structures such as the renal vein.

An additional property of the renal artery that may be of interest is the degree of renal motion relative to the aorta, induced by respiration and/or blood flow pulsatility. A patient's kidney, which located at the distal end of the renal artery, may move as much as 4″ cranially with respiratory excursion. This may impart significant motion to the renal artery connecting the aorta and the kidney, thereby requiring from the neuromodulatory apparatus a unique balance of stiffness and flexibility to maintain contact between the thermal treatment element and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

### IV. Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively. For example, as noted previously, although much of the disclosure herein describes an energy delivery element 24 (e.g., an electrode) in the singular, it should be understood that this disclosure does not exclude two or more energy delivery elements or electrodes.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A method in a computer system having a processor and memory for providing feedback related to a renal neuromodulation treatment, the method comprising:
receiving one or more measures of renal nerve activity;
generating, by the processor, an assessment of the success of a renal neuromodulation treatment and/or a lesion formation operation based, at least in part, on the one or more measures of renal nerve activity; and
providing an indication as to whether the renal neuromodulation treatment and/or the lesion formation operation was within a pre-determined range.

2. The method of claim 1 wherein the one or more measures of renal nerve activity comprise a measure of electrical activity, a measure of a chemical marker, or a physiological measure of renal activity.

3. The method of claim 1, further comprising:
stimulating one or more renal nerves; and
measuring the one or more measures of renal nerve activity after stimulating the one or more renal nerves.

4. The method of claim 3 wherein stimulating the one or more renal nerves comprises providing an electrical, mechanical, and/or chemical stimulus.

5. The method of claim 3 wherein stimulating the one or more renal nerves comprises providing an indirect or direct stimulus.

6. The method of claim 1 wherein providing the indication comprises providing a visual indication and/or an audible indication.

7. The method of claim 6 wherein the visual indication comprises a numeric or text-based indication.

8. The method of claim 6 wherein providing the visual indication comprises activating or modulating a light whose operation indicates whether at least one of the renal neuromodulation treatment and the lesion formation operation was successful.

9. The method of claim 8 wherein modulating a color of the light provides the visual indication.

10. The method of claim 1 wherein a feedback algorithm performs the acts of receiving, generating, and providing.

11. A computer-readable storage medium containing instructions that, when executed by a computer, perform operations comprising:
receiving one or more measures of renal nerve activity;
generating an assessment of the success of a renal neuromodulation treatment and/or a lesion formation operation based, at least in part, on the one or more measures of renal nerve activity; and
providing an indication as to whether the renal neuromodulation treatment and/or the lesion formation operation was within a pre-determined range.

12. The computer-readable storage medium of claim 11 the one or more measures of renal nerve activity comprise a measure of electrical activity, a measure of a chemical marker, or a physiological measure of renal activity.

13. The computer-readable storage medium of claim 11, wherein providing the indication comprises providing one or both of a visual indication or an audible indication.

14. The computer-readable storage medium of claim 13 wherein the visual indication comprises a numeric or text-based indication.

15. The computer-readable storage medium of claim 13 wherein providing the visual indication comprises activating or modulating a light whose operation indicates whether at least one of the renal neuromodulation treatment and the lesion formation operation was successful.

16. A system for intravascular modulation of renal nerves, the system comprising:
a catheter comprising an elongated shaft having a proximal portion and a distal portion, wherein the distal portion comprises an energy delivery element configured for placement within a renal blood vessel;
an energy source coupled to the energy delivery element and configured to deliver energy via the energy delivery element to target neural fibers proximate to a wall of the renal blood vessel,
wherein the energy source further comprises a component configured to—receive one or more measures of renal nerve activity;
generate an assessment of the success of one or both of a renal neuromodulation treatment or a lesion formation operation based, at least in part, on one or more measures of renal nerve activity; and
provide an indication as to whether one or both of the renal neuromodulation treatment or the lesion formation operation was within a pre-determined range.

17. The system of claim 16, further comprising a user interface used to display or play the indication.

18. The system of claim 16 wherein the catheter comprises one or more electrodes configured to deliver electrical energy sufficient for stimulating one or more renal nerves to assess renal nerve function.

19. The system of claim 16 wherein the one or more measures of renal nerve activity comprise at least one of the following: a measure of electrical activity, a measure of a chemical marker, or a physiological measure of renal activity.

20. The system of claim 16 wherein the indication comprises one or both of a visual indication or an audible indication.
